(19) United States
(12) Patent Application Publication
(10) Pub. No.: US 2008/0081057 A1
(43) Pub. Date: Apr. 3, 2008

(54) COMPOSITION HAVING A HEALTHY APPEARANCE EFFECT

(75) Inventor: Veronique CHEVALIER, Villecresnes (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 11/861,326

(22) Filed: Sep. 26, 2007

Related U.S. Application Data
(60) Provisional application No. 60/850,627, filed on Oct. 11, 2006.

(30) Foreign Application Priority Data
Oct. 3, 2006 (FR) 06 54058

(51) Int. Cl. A61K 8/06; A61K 8/33; A61K 8/35; A61Q 1/02; A61Q 19/00

(52) U.S. Cl. 424/401; 424/59; 424/63

(57) ABSTRACT

Composition containing at least one encapsulated pigment and at least one skin colouring agent chosen from self-tanning agents, melanogenesis activators and their mixtures. The composition of the invention constitutes in particular a cosmetic composition which gives an immediate healthy appearance effect which lasts over time.

### REFERENCE TO PRIOR APPLICATIONS

This application claims priority to U.S. provisional application 60/850,627 filed Oct. 11, 2006, and to French patent application 0654058 filed Oct. 3, 2006, both incorporated herein by reference.

### FIELD OF THE INVENTION

The invention relates to compositions for topical use that give a healthy appearance effect to the skin, comprising at least one skin colouring agent (e.g., self-tanning agent and/or melanogenesis activator) and encapsulated pigments.

The invention also relates to the use of the composition to give a healthy appearance effect to the skin and to artificially tan and/or brown the skin.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

In this day and age, it is important to have a healthy appearance and a tanned skin is still a sign of good health. However, natural tanning is not always desirable in so far as it requires prolonged exposure to UV radiation, in particular to UV-A radiation, which causes burning of the skin but which, on the other hand, can induce a detrimental change in the latter, in particular in the case of sensitive skin or of skin continually exposed to solar radiation. It is therefore desirable to find an alternative to natural tanning which is compatible with the requirements of such skin.

The majority of cosmetic products intended for artificial tanning of the skin are based on mono- or polycarbonyl compounds which make possible, by interaction with the amino acids of the skin, the formation of products which will colour the skin. Use may also be made, for this purpose, of melanogenesis activators.

Thus, it is known that dihydroxyacetone or DHA is a particularly advantageous product which is commonly used in cosmetics as agent for the artificial tanning of the skin; applied to the latter, in particular to the face, it makes it possible to obtain a tanning or browning effect with an appearance similar to that which can result from prolonged exposure to the sun (natural tanning) or under a UV lamp.

One disadvantage of DHA is the slowness with which the colouring develops: in fact, several hours (3 to 5 hours in general) are required for the colouring to develop. Moreover, the colouring produced on the skin by DHA is often judged to be excessively yellow by users.

Thus, a search is still being maintained for new compounds and new compositions which make it possible to artificially confer on the skin, in a simple, efficient, rapid and risk-free way, a colouring bestowing on it a healthy appearance effect similar to natural tanning.

It is, of course, known to use make-up products to give a small degree of colour to the skin. The addition of DHA might strengthen or prolong the colouring given by the make-up. However, make-up products are coloured by virtue of the presence of pigments and it is difficult to use make-up products comprising DHA due to the presence of these pigments, in particular iron oxides, which are incompatible with DHA, resulting in the destabilization of the composition. Thus, it is preferable to use the DHA in white products.

### SUMMARY OF THE INVENTION

The need remains for a composition which is not a coloured composition but which, after application to the skin, develops a long-term colour immediately.

The inventor has now discovered such compositions by combining encapsulated pigments with a skin colouring agent, such as a self-tanning agent, such as DHA, and/or a compound which activates melanogenesis.

The compositions according to the invention makes it possible to obtain a colouring of the skin which occurs immediately on application of the composition to the skin and which, in addition, lasts over time. In addition, the use of such a composition exhibits the advantage of completely avoiding the risks of skin reaction generally linked with the abovementioned prolonged exposure (erythema, burns, loss of elasticity, appearance of wrinkles, premature ageing of the skin, and others).

### DETAILED DESCRIPTION OF THE PREFERRED EMBODIMENTS

One subject-matter of the invention is a composition comprising encapsulated pigment(s) and at least one skin colouring agent chosen from self-tanning agents, melanogenesis activators and their mixtures.

The invention compositions are intended for topical application. The term “topical application” is understood here to mean an external application to keratinous substances and in particular skin. As the composition is intended for topical application, it preferably comprises a physiologically acceptable medium. The term “physiologically acceptable medium” is understood to mean a medium compatible with keratinous substances, that is to say the skin, lips, scalp, eyelashes, eyes, nails and/or hair. The composition can in particular constitute a cosmetic or dermatological composition.

The term “encapsulated pigments” is understood to mean, in the present patent application, pigments which are contained in “capsules” constituting a shell around the pigment or pigments. The capsules can comprise a single type of pigment, and thus a single colour, or several types of pigment.

These encapsulated pigments exhibit the advantage of being invisible in the composition by virtue of their encapsulation while being readily released from their capsules when applied to the skin. The pressure exerted during application of the composition to the skin breaks the capsules and the pigment or pigments are released, conferring colour on the skin.

The encapsulated pigments are certainly different from the coated pigments commonly used in make-up products. This is because, whereas coated pigments comprise a chemical coating targeted at improving their dispersion in the composition comprising them, which coating is more or less homogeneous and does not prevent the composition comprising it from being coloured, the encapsulated pigments comprise a physical layer composed of the capsule, this layer being very homogeneous and isolating in sealed fashion the encapsulated pigment from the composition comprising it, so that each encapsulated pigment is completely distinctive in the composition, which is not the case with the coated pigments.

The pigments which are encapsulated can be chosen from inorganic pigments and organic pigments, which may or may not be interference pigments and which may or may not be surface-treated, and their mixtures. The term “pigment” is used to describe any colouring material which is insoluble in the cosmetic starting materials known to a person skilled in the art. They can be alone (a single colour) or as a mixture. The pigments can be of any colour, particularly yellow, black, red, brown, blue and purple.

Mention may in particular be made, as inorganic pigments, of titanium dioxide, zirconium oxides, cerium oxides, zinc oxides, iron oxides (black, yellow or red), chromium oxides, manganese violet, ultramarine blue, chromium hydrate and ferric blue. These pigments can all optionally be surface-treated.

Mention may be made, among organic pigments, of carbon black, pigments of organic lake type of barium, strontium, calcium or aluminium, including those subject to certification by the Food and Drug Administration (FDA) (D&C or FD&C examples) and those exempt from the FDA certification, such as lakes based on cochineal carmine.

The pigments are encapsulated in capsules which have to be both flexible and resistant. Thus, they have to be resistant to the other starting materials present in the composition but flexible enough to be able to burst under shearing when applied to the skin in order thus to deliver the colour desired.

These capsules can, for example, be made of a material which can be chosen from:
jojoba esters,
polymers or copolymers of acrylic acid and/or of methacrylic acid,
cellulose derivatives, such as hydroxypropylmethyl-cellulose,
and their mixtures.

Mention may be made, as pigments encapsulated by capsules made of jojoba esters, for example, of the spheres sold by Floratech (“Spheres of jojoba esters”) under the names Florasomes and described in the document U.S. Pat. No. 6,432,428.

Mention may be made, as pigments encapsulated by capsules made of polymers or copolymers of acrylic acid and/or of methacrylic acid, for example, of the micro-capsules based on acrylates/ammonium methacrylate copolymer sold by Tagra and described in the document WO-A-01/35933.

Mention may be made, as pigments encapsulated by capsules made of cellulose derivatives, for example, of the spheres comprising mannitol, cellulose or hydroxy-propylmethylcellulose sold by Induchem.

Use may also be made, in the same composition, of several types of capsules comprising encapsulated pigments and made of different encapsulation materials.

The capsules can, in addition to the pigments, comprise additives, such as, for example, plasticizing agents and/or opacifying agents, in order for the colour of the encapsulated pigments to be even better concealed. The size of these capsules, that is to say their number-average diameter, is not particularly limited and can range, for example, from 20 to 700 μm.

The percentage of pigments, with respect to the total weight of the encapsulated pigment (weight of the encapsulated pigment=weight of the capsule+weight of the pigments), can vary to a large extent. The amounts of pigments can range, for example, from 10 to 80% by weight, preferably from 20 to 60% by weight and better still from 20 to 50% by weight, with respect to the total weight of the encapsulated pigment.

The composition according to the invention is not particularly limited and can comprise an amount of encapsulated pigments (weight of the capsule+weight of the pigments) ranging, for example, from 0.01 to 20% by weight, preferably from 0.05 to 15% by weight and better still from 0.5 to 10% by weight, with respect to the total weight of the composition.

Apart from the encapsulated pigments, the composition according to the invention comprises a skin colouring agent chosen from self-tanning agents, melanogenesis activators and their mixtures. These agents will slowly colour the skin.

The amount of skin colouring agent(s) in the compositions of the invention is not particularly limited and can range, for example, from 0.05 to 15% by weight, preferably from 0.1 to 10% by weight and better still from 0.5 to 10% by weight, with respect to the total weight of the composition.

Mention may in particular be made, as self-tanning agents, of mono- or polycarbonyl compounds, such as dihydroxyacetone (DHA), isatin, alloxan, ninhydrin, glyceraldehyde, meso-tartraldehyde, glutaraldehyde, erythrulose, pyrozaline-4,5-dione derivatives, such as described in the documents FR-A-2 466 492 and WO-A-97/35842, 4,4-dihydroxypyrazolin-5-one derivatives, such as described in document EP-A-0 903 342, and their mixtures.

In a particularly preferred embodiment of the invention, use is more particularly made, as self-tanning agents, of dihydroxyacetone (DHA), erythrulose and their mixtures.

Use may also be made of mixtures of compounds, which mixtures can mimic the activity of polyphenoloxidase. Thus, in the presence of sodium hydrogencarbonate and of a metal salt in a ratio of 1/50 to 1/200 with an optimum ratio at 1/100, polyphenols will give, by oxidation, a brown colour to the skin or to the hair. Use may thus be made of a mixture comprising a polyphenol, hydrogencarbonate and a metal salt.

Mention may be made, as biological activators of melanogenesis, for example, of:

1) Compounds which stimulate the synthesis of melanin, such as:
a) certain plant extracts, such as rosaceous extracts, in particular of Sanguisorba officinalis, such as described in the document EP-A-993 826, and chrysanthemum (Chrysanthemum) extracts, such as described in the document EP-A-1 014 934, catechins and gallic acid esters of catechins, and also the green tea extracts comprising them, as described in document WO-99/66897;
b) aliphatic or cyclic diols, as described in the paper by Brown D. A. et al., J. Invest. Dermatol., 110, 4, 428-437, 1998;
c) peptides with isoelectric points between 6 and 11, as described in the document WO-A-99/37279;
d) nucleopeptide complexes, as described in the document WO-A-98/12212;
e) adenosine-1 receptor antagonists or adenosine-2 receptor agonists, as described in the document WO-A-98/15276;
f) complexes of metal ions and of peptones, as described in the document U.S. Pat. No. 5,698,184;
g) α-hydroxy acids and their derivatives (for example esters), α-keto acids and their derivatives (for example esters), β-hydroxy acids and their derivatives (for example esters), retinoids, such as vitamin A (retinol) acids, such as trichloroacetic acid and trifluoroacetic acid, phenols and methoxypropyl gluconamide, as described in the document WO-A-99/51198;
h) compounds which stimulate the synthesis of melanin by use of substrates according to the synthetic route: L-tyrosine, L-dihydrophenyl-alanine and L-dopa [see Smit N. P. M. et al., J. Invest. Dermatol., 109, 796-800, 1997].

2) Compounds which stimulate the activity or expression of tyrosinase, such as:
a) the Caesalpinia sappan L. extract described in the document EP-A-0 820 761;
b) Parameria laevigata, Piper cubeba, Sonchus arvensis L., Pluchea indica L., Massoia aromatica Becc., Alstonia scholaris, Alyxia reindwartii Bl., Cinnamomum sintoc Bl., Arctostaphylos, Chenopodium, Poterium or Gaultheria extracts, as described in the document EP-A-0 914 816;
c) prostaglandins, their derivatives, salts and analogues, as described in the document WO-A-98/00100;
d) NO/cGMP/protein kinase G activators, as described in the document WO-A-98/11882;
e) compounds which stimulate the activity or expression of tyrosinase by enhancing the intra-cellular cAMP level, such as:
   proopiomelanocortan peptides, as described in the document WO-A-98/25584;
   cAMP analogues [see McLane et al., Biochem. Biophys. Res. Comm., 145, 719-725, 1987];
   forskolin [see Ortonne J. P., J. Int. Med. Res., 18, 8C-17C, 1990];
   xanthine bases, such as theophylline and caffeine;
f) compounds which stimulate the activity or expression of tyrosinase by activation of protein kinase C:
   diacylglycerols, such as 1-oleyl-2-acetyl-glycerol [see Allan A. E. et al., J. Invest. Dermatol., 105, 5, 687-692, 1995, and the document WO-A-98/55085];
   psoralens [see Anthony F. A. et al., Photo-dermatol. Photoimmunol. Photomed., 1997, 13, 9-16].

3) Compounds which stimulate the transfer of melanosomes from melanocytes to keratinocytes by stimulation of the PAR-2 receptors, as described in the document WO-A-99/04752.

4) And the mixtures of these compounds.

According to a preferred embodiment of the invention, use is made, as melanogenesis activators, of rosaceous extracts, in particular Sanguisorba officinalis extracts, chrysanthemum extracts, α-hydroxy acids and their derivatives, α-keto acids and their derivatives, β-hydroxy acids and their derivatives, retinoids and their derivatives, Caesalpinia sappan L. extract, Parameria laevigata, Piper cubeba, Sonchus arvensis L., Pluchea indica L., Massoia aromatica Becc., Alstonia scholaris, Alyxia reindwartii Bl., Cinnamomum sintoc Bl., Arctostaphylos, Chenopodium, Poterium or Gaultheria extracts, and their mixtures.

The compositions according to the invention can be provided in any formulation form including all those conventionally used for topical application, in particular in the form of aqueous or aqueous/alcoholic solutions, of oil-in-water (O/W) or water-in-oil (W/O) or multiple (triple: W/O/W or O/W/O) emulsions, of aqueous gels, of dehydrated anhydrous products or of dispersions of an oily phase in an aqueous phase using spherules, it being possible for these spherules to be polymeric nanoparticles, such as nanospheres and nanocapsules, or lipid vesicles of ionic type (liposomes) and/or nonionic type. These compositions can be prepared according to standard methods.

In addition, the compositions according to the invention can be more or less fluid and have the appearance of a white or slightly tinted cream, of an ointment, of a milk, of a lotion, of a serum, of a paste or of a foam, for example. They can also be provided in the solid form, for example in the form of a cast stick.

According to a preferred embodiment, the composition is in the form of an emulsion and more particularly of an O/W emulsion.

When the composition according to the invention is an emulsion, it comprises an oily phase. The latter preferably comprises at least one oil, in particular a physiologically acceptable oil. It can additionally comprise other fatty substances.

Mention may be made, as oils which can be used in the composition of the invention, for example, of:
hydrocarbon oils of animal origin, such as perhydrosqualene;
hydrocarbon oils of vegetable origin, such as liquid triglycerides of fatty acids comprising from 4 to 10 carbon atoms, such as triglycerides of heptanoic acid or octanoic acid, or also, for example, sunflower, maize, soybean, cucumber, grape seed, sesame, hazelnut, apricot, macadamia, arara, castor or avocado oils, triglycerides of caprylic/capric acids, such as those sold by Stearineries Dubois or those sold under the names Miglyol 810, 812 and 818 by Dynamit Nobel, jojoba oil or shea butter oil;
synthetic esters and ethers, in particular of fatty acids, such as oils of formulae R¹COOR² and R¹OR² in which R¹ represents the residue of a fatty acid comprising from 8 to 29 carbon atoms and R² represents a linear or branched hydrocarbon chain comprising from 3 to 30 carbon atoms, such as, for example, Purcellin oil, isononyl isononanoate, isopropyl myristate, 2-ethylhexyl palmitate, 2-octyldodecyl stearate, 2-octyldodecyl erucate or isostearyl isostearate; hydroxylated esters, such as isostearyl lactate, octyl hydroxystearate, octyldodecyl hydroxystearate, diisostearyl malate, triisocetyl citrate or heptanoates, octanoates or decanoates of fatty alcohols; polyol esters, such as propylene glycol dioctanoate, neopentyl glycol diheptanoate and diethylene glycol diisononanoate; and pentaerythritol esters, such as pentaerythrityl tetraisostearate;
linear or branched hydrocarbons of mineral or synthetic origin, such as volatile or nonvolatile liquid paraffins and their derivatives, petrolatum, polydecenes or hydrogenated polyisobutene, such as Parleam oil;
fatty alcohols having from 8 to 26 carbon atoms, such as cetyl alcohol, stearyl alcohol and their mixtures (cetearyl alcohol), octyldodecanol, 2-butyloctanol, 2-hexyldecanol, 2-undecylpentadecanol, oleyl alcohol or linoleyl alcohol;
fluorinated oils which partially comprise hydrocarbon and/or silicone, such as those described in the document JP-A-2-295912;
silicone oils, such as volatile or nonvolatile polymethylsiloxanes (PDMSs) comprising a linear or cyclic silicone chain which are liquid or pasty at ambient temperature, in particular cyclopolydimethylsiloxanes(cyclomethicones), such as cyclohexasiloxane; polydimethylsiloxanes comprising pendent alkyl, alkoxy or phenyl groups or alkyl, alkoxy or phenyl groups at the end of the silicone chain, which groups have from 2 to 24 carbon atoms; phenylated silicones, such as phenyl trimethicones, phenyl dimethicones, phenyl(trimethylsiloxy)diphenylsiloxanes, diphenyl dimethicones, diphenyl(methyldiphenyl)trisiloxanes, (2-phenylethyl)trimethylsiloxysilicates and polymethylphenylsiloxanes;
their mixtures.

The term “hydrocarbon oil” is understood to mean, in the list of the oils mentioned above, any oil comprising predominantly carbon and hydrogen atoms and optionally ester, ether, fluorinated, carboxylic acid and/or alcohol groups.

The other fatty substances which can be present in the oily phase are, for example, fatty acids comprising from 8 to 30 carbon atoms, such as stearic acid, lauric acid, palmitic acid and oleic acid; waxes, such as lanolin, beeswax, carnauba wax, candelilla wax, paraffin or lignite waxes or microcrystalline waxes, ceresin or ozokerite, synthetic waxes, such as polyethylene waxes or Fischer-Tropsch waxes; silicone resins, such as trifluoromethyl C1-4 alkyl dimethicone and trifluoropropyl dimethicone; and silicone elastomers, such as the products sold under the “KSG” names by Shin-Etsu, under the “Trefil”, “BY29” or “EPSX” names by Dow Corning or under the “Gransil” names by Grant Industries.

These fatty substances can be chosen in a way varied by a person skilled in the art in order to prepare a composition having the properties, for example of consistency or of texture, desired.

The emulsions generally comprise at least one emulsifier preferably chosen from amphoteric, anionic or nonionic emulsifiers, used alone or as a mixture. The emulsifiers are appropriately chosen according to the continuous phase of the emulsion to be obtained (W/O or O/W). When the emulsion is a multiple emulsion, it generally comprises an emulsifier in the primary emulsion and an emulsifier in the external phase into which the primary emulsion is introduced.

Mention may be made, as emulsifiers which can be used in the preparation of the W/O emulsions, for example, of sorbitan, glycerol or sugar alkyl esters or ethers; silicone surfactants, such as dimethicone copolyols, for example the mixture of cyclomethicone and of dimethicone copolyol sold under the names DC 5225C and DC 3225C by Dow Corning, and such as alkyl dimethicone copolyols, for example the lauryl dimethicone copolyol sold under the name “Dow Corning 5200 Formulation Aid” by Dow Corning, the cetyl dimethicone copolyol sold under the name Abil EM 90® by Goldschmidt and the polyglyceryl-4 isostearate/cetyl dimethicone copolyol/hexyl laurate mixture sold under the name Abil WE 09® by Goldschmidt. It is also possible to add thereto one or more coemulsifiers which can advantageously be chosen from the group consisting of branched-chain fatty acid and polyol esters and in particular branched-chain fatty acid and glycerol and/or sorbitan esters, for example polyglyceryl isostearate, such as the product sold under the name Isolan GI 34 by Goldschmidt, sorbitan isostearate, such as the product sold under the name Arlacel 987 by IC, and sorbitan glyceryl isostearate, such as the product sold under the name Arlacel 986 by ICI, and their mixtures.

Mention may be made, as emulsifiers which can be used in the preparation of the O/W emulsions, for example, of nonionic emulsifiers, such as esters of fatty acids and of oxyalkylene (more particularly polyoxyethylene) polyols, for example polyethylene glycol stearates, such as PEG-100 stearate, PEG-50 stearate and PEG-40 stearate; and their mixtures; oxyalkylenated esters of fatty acids and of sorbitan comprising, for example, from 20 to 100 EO units, for example those sold under the trade names Tween by Uniqema; oxyalkylenated (oxyethylenated and/or oxypropylenated) ethers of fatty alcohols, such as ceteareth-20; sugar esters, in particular sucrose esters, such as sucrose stearate and sucrose tristearate, for example Tegosoft from Degussa, and glucose esters, such as methyl glucose sesquistearate, for example Glucate SS from Noveon; and the mixtures of these emulsifiers, such as, for example, the mixture of glyceryl monostearate and of polyethylene glycol (100 EO) stearate sold under the name Simulsol 165 by Seppic and the mixture of glyceryl stearate and of PEG-100 stearate sold under the name Arlacel 165 by Uniqema.

Coemulsifiers can be added to these emulsifiers, such as, for example, fatty alcohols having from 8 to 26 carbon atoms, such as cetyl alcohol, stearyl alcohol and their mixtures (cetearyl alcohol), octyldodecanol, 2-butyloctanol, 2-hexyldecanol, 2-undecylpentadecanol or oleyl alcohol.

It is also possible to prepare emulsions devoid of emulsifying surfactants or comprising less than 0.5% of the total weight of the composition thereof by using appropriate compounds, for example polymers having emulsifying properties, such as the modified carboxyvinyl polymers sold under the names Carbopol 1342 and Pemulen by Noveon; polymers and copolymers of 2-acrylamido-2-methylpropanesulphonic acid (AMPS) which are optionally crosslinked and/or neutralized, such as the poly(2-acrylamido-2-methylpropanesulphonic acid) sold by Clariant under the name “Hostacerin AMPS” (CTFA name: ammonium polyacryldimethyltauramide) or such as the emulsified polymer sold under the name Sepigel 305 by Seppic (INCI name: Polyacrylamide/C13-C14 Isoparaffin/Laureth-7) or also the AMPS copolymers comprising a hydrophobic chain sold by Clariant under the Aristoflex names; particles formed of ionic or nonionic polymers, more particularly particles formed of anionic polymer, such as in particular polymers of isophthalic acid or of sulphoisophthalic acid, in particular the phthalate/sulphoisophthalate/glycol copolymers (for example diethylene glycol/phthalate/isophthalate/1,4-cyclohexanedimethanol (INCI name: Diglycol/CHDM/Isophthalates/SIP Copolymer)) sold under the names Eastman AQ polymer (AQ35S, AQ38S, AQ55S, AQ48 Ultra) by Eastman Chemical.

It is also possible to prepare emulsions devoid of emulsifiers which are stabilized by silicone particles or particles of metal oxide, such as TiO₂.

In a known way, the composition of the invention can also comprise adjuvants including those standard in the cosmetics or dermatological field, such as hydrophilic or lipophilic gelling agents, hydrophilic or lipophilic active principles, preservatives (for example, phenoxyethanol and parabens), antioxidants, solvents, fragrances, fillers, UV screening agents, bactericides, odour absorbers, colouring materials, salts or polymers. The amount of these various adjuvants are those conventionally used in the field under consideration, for example from 0.01 to 20% of the total weight of the composition. These adjuvants, depending on their nature, can be introduced into the fatty phase, into the aqueous phase and/or into lipid spherules.

Of course, a person skilled in the art will take care to choose the optional additive or additives to be added to the composition according to the invention and their amounts so that the advantageous properties intrinsically attached to the composition in accordance with the invention are not, or not substantially, detrimentally affected by the envisaged addition.

Mention may be made, as fillers which can be used in the composition of the invention, for example, of silica powder; talc; polyamide particles and in particular those sold under the Orgasol name by Atochem; polyethylene powders; powders formed of natural organic materials, such as powders formed of starch, in particular of maize, wheat or rice starches, which may or may not be crosslinked, such as the powders formed of starch crosslinked by octenylsuccinic anhydride sold under the name Dry-Flo by National Starch; microspheres based on acrylic copolymers, such as those made of ethylene glycol dimethacrylate/lauryl methacrylate copolymer sold by Dow Corning under the name of Polytrap; expanded powders, such as hollow microspheres and in particular the microspheres sold under the name Expancel by Kemanord Plast or under the name Micropearl F 80 ED by Matsumoto; silicone resin microbeads, such as those sold under the name Tospearl by Toshiba Silicone; and their mixtures. These fillers can be present in amounts ranging from 0 to 20% by weight and preferably from 1 to 10% by weight, with respect to the total weight of the composition.

One or more hydrophilic or lipophilic gelling agents can in particular be incorporated in the composition according to the invention as polymers. The hydrophilic gelling agents can be chosen, for example, from carboxyvinyl polymers, such as the carbomers sold under the name Carbopol by Noveon; modified carboxyvinyl polymers, such as those sold under the names Carbopol 1342 and Pemulen by Noveon; optionally crosslinked and/or neutralized polymers and copolymers of 2-acryl-amido-2-methylpropanesulphonic acid (AMPS), such as the poly(2-acrylamido-2-methylpropanesulphonic acid) sold by Clariant under the name “Hostacerin AMPS” (CTFA name: Ammonium Polyacryldimethyltauramide) or such as the emulsified polymer sold under the name Sepigel 305 by Seppic (INCI name: Polyacrylamide/C13-C14 Isoparaffin/Laureth-7) or also the AMPS copolymers comprising a hydrophobic chain sold by Clariant under the Aristoflex names; polysaccharides, such as gums and in particular xanthan gum. These gelling agents can be present in an amount ranging, for example, from 0.001 to 10% by weight, preferably from 0.01 to 5% by weight and better still from 0.05 to 3% by weight, with respect to the total weight of the composition.

Mention may be made, as active principles, for example, of:
moisturizing agents, such as, for example, sodium lactate; polyols, such as glycerol, sorbitol or polyethylene glycols; mannitol; amino acids; hyaluronic acid; lanolin; urea and mixtures comprising urea, such as NMF (Natural Moisturizing Factor); petrolatum; N-lauroylpyrrolidonecarboxylic acid and its salts; essential fatty acids; essential oils; hydroxyalkyl-ureas, such as N-(2-hydroxyethyl)urea, sold under the name Hydrovance by National Starch; and their mixtures;
anti-ageing and antiwrinkle active principles and more particularly α-hydroxy acids and in particular acids derived from fruits, such as glycolic acid, lactic acid, malic acid, citric acid, tartaric acid, mandelic acid, their derivatives and their mixtures; β-hydroxy acids, such as salicylic acid and its derivatives, for example 5-(n-octanoyl)salicylic acid or 5-(n-dodecanoyl)salicylic acid; α-keto acids, such as ascorbic acid or vitamin C and its derivatives, such as its salts, for example sodium ascorbate, magnesium ascorbyl phosphate or sodium ascorbyl phosphate; its esters, such as ascorbyl acetate, ascorbyl palmitate and ascorbyl propionate, or its sugars, such as glycosylated ascorbic acid, and their mixtures; β-keto acids; retinoids, such as retinol (vitamin A) and its esters, retinal, retinoic acid and its derivatives, and also the retinoids disclosed in the documents FR-A-2 570 377, EP-A-199 636, EP-A-325 540 and EP-A-402 072; adapalene; carotenoids; and C-glycoside derivatives, such as C-β-D-xylopyranoside-n-propan-2-one, in particular C-β-D-xylopyranoside-n-propan-2-one in the form of a solution, with an active material content of 30%, in a water/propylene glycol mixture (60/40% by weight) manufactured by Chimex under the trade name Mexoryl SBB®;
vitamins, such as vitamin A and vitamin C indicated above and also such as vitamin E (tocopherol) and its derivatives; vitamin B3 (or vitamin PP or niacinamide) and its derivatives; vitamin B5 (or panthenol in its various forms: D-panthenol, DL-panthenol) and its derivatives and analogues, such as calcium pantothenate, pantethine, pantetheine or panthenyl ethyl ether, pangamic acid, pyridoxine, pantoyl lactone and the natural compounds comprising the latter, such as royal jelly; vitamin D and its analogues, such as those disclosed in the document WO-A-00/26167; vitamin F or its analogues, such as mixtures of unsaturated acids having at least one double bond and in particular mixtures of linoleic acid, of linolenic acid and of arachidonic acid, or compounds comprising them;
antibacterials and anti-seborrhoeic agents, such as salicylic acid, 2,4,4′-trichloro-2′-hydroxydiphenyl ether (or triclosan), 3,4,4′-trichlorocarbanilide (or triclocarban), azelaic acid, benzoyl peroxide and zinc salts, such as zinc lactate, gluconate, pidolate, carboxylate, salicylate and/or cysteate.

Mention may be made, as examples of organic screening agents, of the following families: anthranilates, in particular menthyl anthranilate; benzophenones, in particular benzophenone-1, benzophenone-3, benzo-phenone-5, benzophenone-6, benzophenone-8, benzo-phenone-9, benzophenone-12 and preferably benzo-phenone-2 (oxybenzone) or benzophenone-4 (Uvinul MS40, sold by BASF); benzylidenecamphors, in particular 3-benzylidene camphor, benzylidene camphor sulphonic acid, camphor benzalkonium methosulphate, polyacrylamidomethyl benzylidene camphor, terephthalylidene dicamphor sulphonic acid and 4-methylbenzylidene camphor (Eusolex 6300, sold by Merck); benzimidazoles, in particular benzimidazilate (Neo Heliopan AP, sold by Haarmann and Reimer) or phenylbenzimidazole sulphonic acid (Eusolex 232, sold by Merck); benzotriazoles, in particular drometrizole trisiloxane or methylene bis-benzotriazolyl tetramethylbutylphenol (Tinosorb M, sold by Ciba); cinnamates, in particular cinoxate, DEA methoxycinnamate, diisopropyl methyl cinnamate, glyceryl ethylhexanoate dimethoxycinnamate, isopropyl methoxycinnamate, isoamyl cinnamate and preferably ethocrylene (Uvinul N35, sold by BASF), octyl methoxycinnamate (Parsol MCX, sold by Hoffmann-LaRoche) or octocrylene (Uvinul 539, sold by BASF); dibenzoylmethanes, in particular butyl methoxy-dibenzoylmethane (Parsol 1789); imidazolines, in particular ethylhexyl dimethoxybenzylidene dioxoimidazoline propionate; PABAs, in particular ethyl dihydroxypropyl PABA, ethylhexyl dimethyl PABA, glyceryl PABA, PABA, PEG-25 PABA and preferably diethylhexyl butamido triazone (Uvasorb HEB, sold by 3V Sigma), ethylhexyl triazone (Uvinul T150, sold by BASF) or ethyl PABA (benzocaine); salicylates, in particular dipropylene glycol salicylate, ethylhexyl salicylate, homosalate or TEA salicylate; or triazines, in particular anisotriazine (Tinosorb S, sold by Ciba).

The amount of screening agents depends on the final use desired. It can range, for example, from 1 to 20% by weight and better still from 2 to 10% by weight, with respect to the total weight of the composition.

The composition of the invention preferably bestows an immediate healthy appearance effect and, in addition, this effect preferably lasts over time. It can constitute in particular a cosmetic composition for the care of the skin contributing this healthy appearance effect which lasts over time.

The invention also relates to a method for the cosmetic treatment of the skin in order to give it a healthy appearance effect, characterized in that it comprises applying, to the skin, an effective amount of a composition as described above. Generally, the effective amount of composition applied to the skin ranges from 0.5 to 4 mg/cm², better still from 1 to 3 mg/cm² and even better still from 1 to 2 mg/cm², but is variable in view of skin type, desired results, etc.

The following examples illustrate the invention without exhibiting a limiting nature. The amounts therein are percentages by weight. The compounds are indicated by INCI name or by chemical name, as the case may be.

### EXAMPLE 1

### O/W Emulsion

Oily phase

| | |
|---|---|
| Apricot oil | 6% |
| Cyclohexasiloxane | 10% |
| Stearyl alcohol (and) ceteareth-20 (Ritapro | 2% |
| 200 from Rita) | |

Aqueous phase

| | |
|---|---|
| Methyl glucose sesquistearate (Glucate SS) | 2% |
| Xanthan gum | 0.25% |
| Glycerol | 5% |
| Preservatives | 0.6% |
| Water | q.s. for 100% |
| Aluminium starch octenylsuccinate (Dry Flo) | 3% |
| Encapsulated pigments (Tagra microcapsules) | 1.5% |
| Sanguisorba officinalis extract (Burnet | 1% |
| extract powder from Maruzen) | |
| DHA | 5% |

Procedure: The oily phase was heated at approximately 80° C. until completely dissolved and then it was introduced into the aqueous phase, brought beforehand to the same temperature, to form an O/W emulsion, to which the pigments and then the starch derivative, Sanguisorba extract and the DHA were added.

A fluid emulsion was obtained which is suitable for providing, when applied to the skin, an immediate healthy appearance effect which lasts over time. Furthermore, the emulsion has good moisturizing properties.

### EXAMPLE 2

### O/W Emulsion

Oily phase

| | |
|---|---|
| Apricot oil | 7% |
| Cyclohexasiloxane | 6% |
| Caprylic/capric triglyceride | 7% |
| Stearic acid | 1% |
| Polysorbate 61 (Tween 61 V) | 1.35% |

Aqueous phase

| | |
|---|---|
| Sucrose tristearate (Tegosoft) | 2% |
| Xanthan gum | 0.25% |
| Carbomer | 0.3% |
| Glycerol | 5% |
| Propylene glycol | 1% |
| Preservatives | 1.1% |
| C-β-D-Xylopyranoside-n-propan-2-one | 5% |
| (Mexoryl SBB) | |
| Water | q.s. for 100% |
| Aluminium starch octenylsuccinate (Dry Flo) | 3% |
| Erythrulose | 5% |
| Chrysanthemum extract (*) | 5% |
| Encapsulated pigments (Florasomes) | 1% |

(*) The chrysanthemum extract was obtained according to protocol 1 described in EP-A-1 014 934: the chrysanthemum powder was mixed with an aqueous extraction solvent composed of cell culture medium DMEM F-12 3:1, sold by Life Technologies, in a proportion of concentration of 5 grams of dry powder per 100 ml of solvent. The mixture was kept stirred at ambient temperature for 4 hours. The mixture was then centrifuged at 1000 revolutions/minute for 8 minutes and the supernatant was withdrawn and subjected to two identical centrifuging/withdrawal cycles. The final supernatant withdrawn was filtered through a 0.22 μm filter of Millipore type under aseptic conditions in order to be sterilized and was stored at a temperature of 4° C. until use.

Procedure for the preparation of the example: The oily phase was heated at approximately 80° C. until completely dissolved and then it was introduced into the aqueous phase, brought beforehand to the same temperature, to form an O/W emulsion, to which the pigments and then the starch derivative, the erythrulose and the chrysanthemum extract were added.

An anti-ageing composition was obtained which makes it possible to render the complexion uniform and which is particularly suitable for mature skin.

### EXAMPLE 3

### Care Cream (O/W Emulsion)

Oily phase

| | |
|---|---|
| Glyceryl stearate/PEG-100 stearate | 2.5% |
| (Arlacel 165) | |
| Polysorbate 60 (Tween 60 V) | 2.5% |
| Cetyl alcohol | 1% |
| Stearyl alcohol | 1% |
| Paraffin | 5% |

Aqueous phase

| | |
|---|---|
| Carbomer | 0.3% |
| Base (sodium hydroxide) | 0.2% |
| Preservative | 0.2% |
| N-(2-Hydroxyethyl) urea (Hydrovance) | 5% |
| Water | q.s. for 100% |
| Nylon 66 fibres | 2% |
| Sanguisorba officinalis extract (Burnet | 5% |
| extract powder from Maruzen) | |
| Encapsulated pigments (Tagra microcapsules) | 2% |

Procedure: The oily phase was heated at approximately 80° C. until completely dissolved and then it was introduced into the aqueous phase, brought beforehand to the same temperature, to form an O/W emulsion, to which the pigments and then the nylon fibres and the Sanguisorba extract were added.

A skincare cream was obtained which gives a tanned and satin-smooth appearance and which is suitable for moisturizing the skin and for masking dyschromias, immediately and over time.

### EXAMPLE 4

### Care Cream (O/W Emulsion)

Oily phase

| | |
|---|---|
| Apricot oil | 7% |
| Cyclohexasiloxane | 6% |
| Caprylic/capric triglyceride | 7% |
| Stearic acid | 1% |
| Polysorbate 61 (Tween 61 V) | 1.35% |
| Sucrose tristearate | 2% |

Aqueous phase

| | |
|---|---|
| Xanthan gum | 0.25% |
| Aluminium starch octenylsuccinate (Dry-Flo) | 3% |
| Carbomer | 0.3% |
| Glycerol | 5% |
| Propylene glycol | 1% |
| Preservatives | 1.1% |
| Erythrulose | 5% |
| Chrysanthemum extract* | 5% |
| Pigments encapsulated in waxes formed of | 1% |
| jojoba esters | |
| Water | q.s. for 100% |

(*lyophilisate of aqueous extract of Chrysanthemum sinensis leaves)

Procedure:

The oily phase and the aqueous phase are heated separately to a temperature of 80° C. The oily phase is poured into the aqueous phase while stirring at 4000 rev/min, the stirring being provided by a Moritz homogenizer of Turbo Lab 2100 type, and the stirring and temperature conditions are retained for 30 minutes. The mixture is then introduced into a Soavi high-pressure homogenizer of OBL type adjusted to a pressure of 500 bar for 3 consecutive passes. The product is then cooled until it has returned to ambient temperature.

An oil-in-water emulsion is thus obtained, the oily globules of which have a mean size of less than 200 nm and a polydispersity with an index of less than 0.1, as measured by a laser particle sizer of Amtech BI 90 type.

The above written description of the invention provides a manner and process of making and using it such that any person skilled in this art is enabled to make and use the same, this enablement being provided in particular for the subject matter of the appended claims, which make up a part of the original description and a composition comprising encapsulated pigments and at least one skin colouring agent chosen from self-tanning agents, melanogenesis activators and their mixtures.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A composition comprising at least one encapsulated pigment and at least one skin colouring agent chosen from self-tanning agents, melanogenesis activators and mixtures thereof.

2. The composition according to claim 1, wherein the pigment is chosen from inorganic pigments and organic pigments, which may or may not be interference pigments and which may or may not be surface-treated, and mixtures thereof.

3. The composition according to claim 1, wherein the pigment is encapsulated in capsules comprising a material chosen from jojoba esters, polymers or copolymers of acrylic acid and/or of methacrylic acid, cellulose derivatives, and mixtures thereof.

4. The composition according to claim 3, wherein the capsules have a size ranging from 20 to 700 μm.

5. The composition according to claim 1, wherein the pigment is present in an amount ranging from 10 to 80% by weight with respect to the total weight of the encapsulated pigment.

6. The composition according to claim 1, comprising an amount of encapsulated pigments ranging from 0.01 to 20% by weight with respect to the total weight of the composition.

7. The composition according to claim 1, wherein the amount of skin colouring agent(s) ranges from 0.05 to 15% by weight with respect to the total weight of the composition.

8. The composition according to claim 1, comprising at least one self-tanning agent selected from mono- and polycarbonyl compounds, and mixtures thereof.

9. The composition according to claim 1, comprising at least one self-tanning agent chosen from dihydroxyacetone, erythrulose and mixtures thereof.

10. The composition according to claim 1, comprising at least one biological activator of melanogenesis chosen from compounds which stimulate the synthesis of melanin, compounds which stimulate the activity or expression of tyrosinase, compounds which stimulate the transfer of melanosomes from melanocytes to keratinocytes by stimulation of the PAR-2 receptors, and mixtures thereof.

11. The composition according to claim 1, comprising at least one biological activator of melanogenesis chosen from rosaceous extracts, chrysanthemum extracts, α-hydroxy acids and their derivatives, α-keto acids and their derivatives, β-hydroxy acids and their derivatives, retinoids and their derivatives, Caesalpinia sappan L. extract, Parameria laevigata, Piper cubeba, Sonchus arvensis L., Pluchea indica L., Massoia aromatica Becc., Alstonia scholaris, Alyxia reindwartii Bl., Cinnamomum sintoc Bl., Arctostaphylos, Chenopodium, Poterium or Gaultheria extracts, and mixtures thereof.

12. The composition according to claim 1, wherein the composition is in the form of an emulsion.

13. The composition according to claim 1, wherein the composition is a cosmetic composition for the care of the skin.

14. The composition according to claim 1, comprising at least one self-tanning agent selected from dihydroxyacetone, isatin, alloxan, ninhydrin, glyceraldehyde, meso-tartraldehyde, glutaraldehyde, erythrulose, pyrozaline-4,5-dione derivatives, 4,4-dihydroxypyrazolin-5-one derivatives, and mixtures thereof.

15. A method for the treatment of the skin in order to give it a healthy appearance effect, comprising applying, to the skin, an effective amount of a composition according to claim 1.
